(19) United States
(12) Patent Application Publication
(10) Pub. No.: US 2008/0027034 A1
(43) Pub. Date: Jan. 31, 2008

(54) Ciclesonide and Syk Inhibitor Combination and Method of Use Thereof

(76) Inventors: Tushar P. Shah, Flemington, NJ (US); Shahin Sanjar, Far Hills, NJ (US); Pamela Weir, Wharton, NJ (US)

Correspondence Address: NATH & ASSOCIATES PLLC, 112 South West Street, Alexandria, VA, 22314, US

(21) Appl. No.: 11/661,150

(22) PCT Filed: Sep. 8, 2005
(86) PCT No.: PCT/EP05/54468

§ 371 Date: Apr. 26, 2007

Related U.S. Application Data
(60) Provisional application No. 60/608,542, filed on Sep. 10, 2004.

(51) Int. Cl. A61K 31/58; A61P 11/00; A61P 11/06; A61P 11/08

(52) U.S. Cl. 514/171; 514/174

(57) ABSTRACT

The invention relates to pharmaceutical formulations containing combinations of ciclesonide and a syk inhibitor and the use of such pharmaceutical compositions in medicine, in particular in the prophylaxis and treatment of respiratory disease.

### FIELD OF THE INVENTION

This invention relates to the combination of ciclesonide with a syk inhibitor, in particular to pharmaceutical formulations containing combinations of ciclesonide and a syk inhibitor and methods comprising the simultaneous or sequential administration of a combination of ciclesonide and a syk inhibitor, in particular methods for the prophylaxis and treatment of allergic and respiratory diseases.

### BACKGROUND

U.S. Pat. No. 6,432,963 and U.S. Patent Application Publication No. US2004/0029902 A1 both disclose selective syk inhibitors and their use in methods for the treatment of inflammatory and allergic conditions associated with Fc receptor signalling in which the syk kinase takes part.

U.S. Pat. No. 5,733,901 discloses pregna-1,4-diene-3,20-dione-16-17-acetal-21 esters and their use in the treatment of inflammatory conditions. The compounds have the general structure: 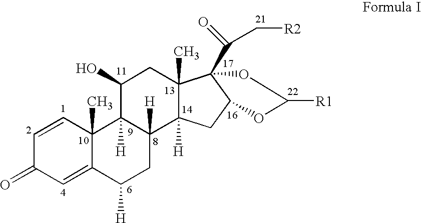
wherein R1 is 2-propyl, 1-butyl, 2-butyl, cyclohexyl or phenyl; and R2 is acetyl or isobutanoyl. Ciclesonide is the INN for a compound of formula I in which R1 is cyclohexyl and R2 is isobutanoyl with the chemical name [11β,16α(R)]-16,17-[(Cyclohexylmethylen)bis(oxy)]-11-hydroxy-21-(2-methyl-1-oxoprop-oxy)pregna-1,4-dien-3,20-dione.

This compound has undergone evaluation as an antiasthmatic and pharmacokinetic studies show that it will be useful in an inhaler formulation. Ciclesonide is only moderately absorbed after oral administration and has low systemic activity. Concentration of the drug in the lungs is high and metabolism by liver oxidases is very high, giving the drug a low plasma half-life. Systemic activity of ciclesonide is three times lower than that of budesonide, but anti-inflammatory activity is higher for the former.

### SUMMARY OF THE INVENTION

It is now surprisingly found that by combined administration of a syk inhibitor and ciclesonide significant unexpected therapeutic benefit is obtained. Without being bound by theory, the improvement in the therapeutic benefit by combining ciclesonide and a syk inhibitor is believed to be based on the different mechanism of action of each agent, which leads to the control of central components of an allergic and/or inflammatory reaction. An allergic reaction is recognized by an acute degranulation and release of preformed mediators such as histamine and tryptase from mast cell or basophils. Subsequent to the acute response, there is a delayed but sustained release of other inflammatory mediators such as cytokines or chemokines that are synthesized as a result of activation of relevant genes in the activated cells.

Inhaled corticosteroid have no effect on the acute degranulation phase of mast cells or basophils hence they are not able to inhibit the release of histamine or tryptase. On the other hand, syk inhibitors, by virtue of inhibiting an important step in the IgE signalling pathway, are able to prevent acute cellular degranulation. The sustained phase of an allergic reaction, as exemplified by cytokine or chemokine release, can be inhibited by both agents but by different mechanisms. Corticosteroids such as ciclesonide achieve their anti-inflammatory effect by entering into the cell cytoplasm and binding to a specific receptor. This corticosteroid/receptor complex inhibits the actions of transcription factors such as AP1 or NFkB, which are responsible to switching on a number of inflammatory cytokine and chemokine genes and so initiate the synthesis and release of these mediators. In addition, the dimeric form of the corticosteroid/receptor complex can directly bind with DNA and alter expression of inflammatory genes. Syk inhibitors also inhibit cytokine or chemokine release from activated inflammatory cells, however, this mechanism is distinct from that which is exerted by ICS and involves intracellular pathways that are currently under investigation.

Thus, based on the complementary action of syk inhibitors and inhaled corticosteroids on acute and sustained phases of an allergic reaction, and their prevention of the inflammatory response at different control points in the inflammatory process, the combination of inhibitors of these pathways provides a broader and more complete anti-inflammatory effect and so leads to greater therapeutic benefit.

Thus in one aspect the present invention relates to a pharmaceutical formulation comprising a syk inhibitor in combination with ciclesonide, a pharmaceutically acceptable salt, solvent or physiologically functional derivative thereof.

Ciclesonide (hereinafter also referred to as active ingredient) is the INN for a compound with the chemical name [11β,16α(R)]-16,17-[(Cyclohexylmethylen)bis(oxy)]-11-hydroxy-21-(2-methyl-1-oxoprop-oxy)pregna-1,4-dien-3,20-dion. Ciclesonide and its preparation are disclosed in DE 4129535. Ciclesonide as used herein also includes, pharmaceutically acceptable salts of ciclesonide, epimers of ciclesonide (e.g. [11β,16α(S)]-16,17-[(Cyclohexylmethylen)bis(oxy)]-11-hydroxy-21-(2-methyl-1-oxopropoxy)-pregna-1,4-dien-3,20-dion) in any mixing ratio with ciclesonide, solvates of ciclesonide, physiologically functional derivatives of ciclesonide or solvates thereof. By the term “physiologically functional derivative” is meant a chemical derivative of ciclesonide having the same physiological function as ciclesonide, for example, by being convertible in the body thereto or by being an active metabolite of ciclesonide. Physiological functional derivatives of ciclesonide which may be mentioned in connection with the invention are for example the 21-hydroxy derivative of ciclesonide with the chemical name 16α,17-(22R,S)-Cyclohexylmethylendioxy-11β,21-di-hydroxypregna-1,4-dien-3,20-dion, in particular 16α,17-(22R)-Cyclohexylmethylendioxy-11β,21-di-hydroxypregna-1,4-dien-3,20-dion. This compound and its preparation are disclosed in U.S. Pat. No. 5,733,901, which is hereby incorporated by reference in its entirety.

Syk inhibitors, as employed in the present invention, include those compounds disclosed in U.S. Pat. No. 6,432,963, which patent is hereby incorporated by reference in its entirety; emphasized may be those compounds encompassed by the definition set out between column 3, line 45 to column 6, line 22, and in particular a compound selected from the group consisting of 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromo-anilino)pyrimidine-5-carboxamide, 2-(cis-2-aminocyclohexylamino)-4-(3,5-dichloroanilino)pyrimidine-5-carboxamide and 2-(cis-2-aminocyclohexylamino)-4-(3,4,5-trimethoxyanilino)pyrimidine-5-carboxamide or a salt thereof. Methods for the synthesis of such compounds are set forth between column 6, line 43 to column 13, line 17.

Syk inhibitors, as employed in the present invention, also include those compounds disclosed in U.S. Patent Application Publication No. US2004/0029902 A1, published on Feb. 12, 2004, inventors R. Singh et al, which patent application publication is hereby incorporated by reference in its entirety; emphasized may be those compounds encompassed by the definition set out between paragraphs 0109 and 0218, and in particular a compound selected from the group consisting of
N2,N4-[(2,2-Dimethyl-4H-benzo[1,4]oxazin-3-one)-6-yl]-5-fluoro-2,4-pyrimidinediamine,
N4-(3,4-Dichlorophenyl)-5-fluoro-N2-(indazoline-6-yl)-2,4-pyrimidinediamine,
N4-(3,4-Ethylenedioxyphenyl)-5-fluoro-N2-(1-methyl-indazoline-5-yl)-2,4-pyrimidinediamine,
N2,N4-Bis(3-hydroxyphenyl)-5-fluoro-2,4-pyrimidinediamine,
N2,N4-Bis(3,4-ethylenedioxyphenyl)-5-fluoro-2,4-pyrimidinediamine,
N4-(1,4-Benzoxazin-6-yl)-5-fluoro-N2-[3-(N-methylamino)carbonylmethyleneoxyphenyl]-2,4-pyrimidinediamine,
N2,N4-Bis(3-aminophenyl)-5-fluoro-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N4-(3-Hydroxyphenyl)-5-trifluoromethyl-N2-[3-(N-methylamino)carbonylmethyleneoxyphenyl]-2,4-pyrimidinediamine,
5-Fluoro-N4-[(1H)-indol-6-yl]-N2-[3-(N-methylamino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
5-Fluoro-N2-(3-methylaminocarbonylmethyleneoxyphenyl)-N4-[2-H-pyrido[3,2-b]-1,4-oxazin-3(4H)-one-6-yl]-2,4-pyrimidinediamine,
N4-(3,4-Ethylenedioxyphenyl)-5-fluoro-N2-[3-(2-hydroxyethyl-amino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N2,N4-Bis(indol-6-yl)-5-fluoro-2,4-pyrimidinediamine,
5-Fluoro-N2-[2-(2-hydroxy-1,1-dimethylethylamino)carbonylbenzofuran-5-yl]-N4-(3-hydroxyphenyl)-2,4-pyrimidinediamine,
N2-[3-(N2,3-Dihydroxypropylamino)carbonylmethyleneoxyphenyl]-N4-(3,4-ethylenedioxyphenyl)-5-fluoro-2,4-pyrimidinediamine,
N2-(3,5-Dimethoxyphenyl)-N4-(3,4-ethylenedioxyphenyl)-5-fluoro-2,4-pyrimidinediamine,
N4-(3,4-Ethylenedioxyphenyl)-5-fluoro-N2-[3-(1,3-oxazol-5-yl)phenyl]-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N2-(3-hydroxyphenyl)-N4-[4-(3-phenyl-1,2-4-oxadiazol-5-yl)methyleneoxyphenyl]-2,4-pyrimidinediamine,
N4-(3,4-Ethylenedioxyphenyl)-5-fluoro-N2-(indazolin-6-yl)-2,4-pyrimidinediamine,
5-Fluoro-N4-(3-hydroxyphenyl)-N2-(indazolin-6-yl)-2,4-pyrimidinediamine,
N4-(3,4-Ethylenedioxyphenyl)-5-fluoro-N2-(1-methyl-indazoline-5-yl)-2,4-pyrimidinediamine,
5-Fluoro-N4-(3-hydroxyphenyl)-N2-(1-methy-indazoline-5-yl)-2,4-pyrimidinediamine,
N4-(3,4-Ethylenedioxyphenyl)-5-fluoro-N2-[4-(3-phenyl-1,2,4-oxadiazol-5-yl)methyleneoxyphenyl]-2,4-pyrimidinediamine,
N4-(3,5-Dimethyl-4-hydroxyphenyl)-5-fluoro-N2-[3-[2-(N-morpholino)ethyleneoxy]phenyl]-2,4-pyrimidinediamine,
N4-(3,5-Dimethyl-4-hydroxyphenyl)-5-fluoro-N2-[3-[2-(N-morpholino)ethyloxy]phenyl]-2,4-pyrimidinediamine,
N4-(3-Chloro-4-hydroxy-5-methylphenyl)-5-fluoro-N2-[3-[2-(N-morpholino)ethyloxy]phenyl]-2,4-pyrimidinediamine,
N2-(3-tert-Butylcarbonylaminophenyl)-N4-(3-hydroxyphenyl)-5-fluoro-2,4-pyrimidinediamine,
N4-(3-tert-Butylphenyl)-N2-[3-(N-methylamino)carbonylmethyleneoxyphenyl]-5-fluoro-2,4-pyrimidinediamine,
N4-(3-tert-Butylphenyl)-N2-[3-(N2,3-dihydroxypropylamino)carbonylmethyleneoxyphenyl]-5-fluoro-2,4-pyrimidinediamine,
N2-[3-(N2,3-Dihydroxypropylamino)carbonylmethyleneoxyphenyl]-5-fluoro-N4-(3-isopropylphenyl)-2,4-pyrimidinediamine,
N4-[4-(Cyanomethyleneoxy)phenyl]-5-fluoro-N2-(3-hydroxyphenyl)-2,4-pyrimidinediamine,
N4-(3,5-Dimethyl-4-hydroxyphenyl)-5-fluoro-N2-[3-(N-piperazino)carbonylmethyleneoxyphenyl]-2,4-pyrimidinediamine,
N4-(3,5-Dimethyl-4-hydroxyphenyl)-5-fluoro-N2-[3-[2-(N-piperazino)ethoxy]phenyl]-2,4-pyrimidine-diamine bis hydrogenchloride salt,
N4-(3,4-Ethylenedioxyphenyl)-5-fluoro-N2-[4-(2-hydroxyethyloxy)phenyl]-2,4-pyrimidinediamine,
N4-(1,4-Benzoxazine-3-on-6-yl)-5-fluoro-N2-(3-hydroxyphenyl)-2,4-pyrimidinediamine,
(+/−)-5-Fluoro-N2-[(N-methylacetamido-2)-3-phenoxy]-N4-(2-methyl-1,4-benzoxazin-6-yl)-2,4-pyrimidinediamine,
N2-(1,4-Benzoxazin-3-on-6-yl)-5-fluoro-N4-(3-hydroxyphenyl)-2,4-pyrimidinediamine,
N4-(3-Chloro-4-trifluoromethoxyphenyl)-5-fluoro-N2-[3-(N-methylamino)carbonylmethyleneoxyphenyl]-2,4-pyrimidinediamine,
5-Fluoro-N4-(3-hydroxy-4-methylphenyl)-N2-[3-[(N-methylamino)carbonylmethyleneoxy]phenyl]-2,4-pyrimidinediamine,
5-Fluoro-N4-(3-hydroxyphenyl)-N2-[4-methyl-3-[(N-methylamino)carbonylmethyleneoxy]phenyl]-2,4-pyrimidinediamine,
5-Fluoro-N4-(3-hydroxy-4-methoxyphenyl)-N2-[3-(N-methylamino)carbonylmethyleneoxyphenyl]-2,4-pyrimidinediamine,
N4-(3-Chloro-4-methylphenyl)-5-fluoro-N2-[3-(N-methylamino)-carbonylmethyleneoxyphenyl]-2,4-pyrimidinediamine,
N4-(3-Chloro-4-methoxyphenyl)-5-fluoro-N2-[3-[(N-methylamino)carbonylmethyleneoxy]phenyl]-2,4-pyrimidinediamine,
5-Fluoro-N4-1(1H)-indol-5-yl]-N2-[3-[(N-methylamino)carbonylmethyleneoxy]phenyl]-2,4-pyrimidinediamine,
5-Fluoro-N4-(3-hydroxyphenyl)-N2-[1-(methoxycarbonyl)methyl-indazoline-5-yl]-2,4-pyrimidinediamine,
5-Fluoro-N4-(3-hydroxyphenyl)-N2-[1-(3-hydroxypropyl)indazoline-6-yl]-2,4-pyrimidinediamine,
N4-(3,4-Ethylenedioxyphenyl)-5-fluoro-N2-[1-(3-hydroxypropyl)indazoline-5-yl]-2,4-pyrimidinediamine,
5-Fluoro-N4-(3-hydroxyphenyl)-N2-[1-(3-hydroxypropyl)indazoline-5-yl]-2,4-pyrimidinediamine,
5-Fluoro-N2-[1-(3-hydroxypropyl)indazoline-5-yl]-N4-(4-isopropoxyphenyl)-2,4-pyrimidinediamine,
N4-(3,4-Ethylenedioxyphenyl)-5-fluoro-N2-[1-[2(N-methylaminocarbonyl)ethyl]-indazoline-5-yl]-2,4-pyrimidinediamine,
5-Fluoro-N4-(4-isopropoxyphenyl)-N2-[1-[2(N-methylaminocarbonyl)ethyl]-indazoline-5-yl]-2,4-pyrimidinediamine,
N4-[(2,2-dimethyl-4H-benzo[1,4]oxazin-3-one)-6-yl]-5-fluoro-N2-[3-(methylaminocarbonylmethylene-oxy)phenyl]-2,4-pyrimidinediamine,
N4-[(2,2-Dimethyl-4H-benzo[1,4]oxazin-3-one)-6-yl]-5-fluoro-N2-(1-methylindazolin-5-yl)-2,4-pyrimidinediamine,
N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine,
N4-1 (2,2-Dimethyl-4H-5-pyridol-1,4]oxazin-3-one)-6-yl]-5-fluoro-N2-[3-(methylaminocarbonyl-methyleneoxy)phenyl]-2,4-pyrimidinediamine,
5-Fluoro-N2-(3-methylaminocarbonylmethyleneoxyphenyl)-N4-[2H-pyrido[3,2-b]-1,4-oxazin-3(4H)-one-6-yl]-2,4-pyrimidinediamine,
N4-(4-Amino-3,4-dihydro-2H-1-benzopyran-6-yl)-5-fluoro-N2-[3-(N-methylamino)carbonylmethyleneoxyphenyl]-2,4-pyrimidinediamine,
N4-(3-Chloro-4-hydroxy-5-methylphenyl)-5-fluoro-N2-[3-[2-(N-piperazino)ethoxy]phenyl]-2,4-pyrimidinediamine, and
N4-(3-Methylcarbonyloximephenyl)-5-fluoro-N2-[3-(N-methylamino)carbonylmethyleneoxyphenyl]-2,4-pyrimidinediamine or a salt thereof.

Such compounds can be synthesized, e.g., by methods set out between paragraphs 0218 and 0260 of U.S. Patent Application Publication No. US2004/0029902.

It will be appreciated that the compounds of the combination may be administered simultaneously, either in the same pharmaceutical formulation (hereinafter also referred to as fixed combination) or in different pharmaceutical formulations (hereinafter also referred to as free combination) or sequentially in any order. If there is sequential administration, the delay in administering the second compound should not be such as to lose the beneficial therapeutic effect of the combination. As an example, both drugs may be provided separately as oral formulations, or one may be an oral preparation and the other an inhalant or topical nasal preparation, or both may be provided in a form suitable for inhalation or topical nasal administration. Administration may be simultaneous or sequential, and sequential administration can be either close in time or remotely, such as where one drug is administered in the morning and the second drug is administered in the evening.

As mentioned above, both syk inhibitors and ciclesonide and their pharmaceutically acceptable salts, solvates, and physiologically functional derivatives have been described for use in the treatment of respiratory diseases. Therefore, formulations of a syk inhibitor and ciclesonide, pharmaceutically acceptable salts, solvates, and physiologically functional derivatives have use in the prophylaxis and treatment of clinical conditions for which a corticosteroid and/or a syk inhibitor is indicated. Such conditions include diseases associated with reversible or irreversible or partially reversible airways obstruction such as asthma, nocturnal asthma, exercise-induced asthma, chronic obstructive pulmonary diseases (COPD) (e. g. chronic and wheezy bronchitis, emphysema, shortness of breath), respiratory tract infection and upper respiratory tract disease (e. g. rhinitis, such as allergic and seasonal rhinitis). The combination may be administered prophylactically or after onset of symptoms.

Accordingly, the present invention also provides a method for the prophylaxis or treatment of a clinical condition in a mammal, such as a human, for which a corticosteroid and/or a syk inhibitor is/are indicated, which comprises administration of a therapeutically effective amount of a pharmaceutical formulation comprising ciclesonide or a pharmaceutical acceptable salt, solvate, or physiologically functional derivative thereof and a a syk inhibitor, and a pharmaceutical acceptable carrier and/or one or more excipients. In a preferred aspect, there is provided such a method, which comprises administration of a therapeutically effective amount of a combination comprising ciclesonide and a syk inhibitor and a pharmaceutical acceptable carrier and/or one or more excipients. In particular, the present invention provides such a method for the prophylaxis or treatment of a disease associated with reversible airways obstruction such as asthma, chronic obstructive pulmonary disease (COPD), respiratory tract infection, or upper respiratory tract disease (e.g., allergic or seasonal rhinitis).

The amount of ciclesonide or a pharmaceutical acceptable salt, solvate or physiologically functional derivative thereof and a syk inhibitor which is required to achieve a therapeutic effect will, of course, vary with the particular compound, the route of administration, the subject under treatment, and the particular disorder or disease being treated.

As a monotherapy, ciclesonide is generally administered to adult humans by inhalation at a daily dose of from 0.05 to 2 mg, which can be administered in one or several doses.

The dosage of the pharmaceutically acceptable salt of a syk inhibitor is in the order of magnitude customary for a syk inhibitor for the treatment of respiratory diseases for example in doses between about 0.0001 and 100 mg/kg per day, e.g., 0.0001 mg/kg/day, 0.001 mg/kg/day, 0.01 mg/kg/day, 0.1 mg/kg/day, 1 mg/kg/day, 10 mg/kg/day and 100 mg/kg/day. Doses of syk inhibitor can of course be higher or lower depending on the age of the patient, condition, bioavailability of the inhibitor, and mode of administration.

It is preferred in connection with the present invention to have a twice daily and particularly preferred to have a once daily dosing regimen.

Suitably, the pharmaceutical formulations for inhalation according to the invention comprise the active ingredients in amounts such that in case of administration by inhalation from inhalers each actuation provides a therapeutically effective dose, for example, a dose of ciclesonide of 10 μg to 800 μg, 25 μg to 400 μg, preferably 50 μg to 200 μg (e.g. 100 μg) and a dose of a syk inhibitor or a pharmaceutically acceptable salt thereof in a range between about 0.0001 and 100 mg/kg per day. It is particularly preferred that each actuation provide a dose therapeutically effective for a twice-daily dosing regimen or more particularly preferred for a once daily dosing regimen.

Suitably, the pharmaceutical formulations for inhalation according to the invention provide therapeutically effective doses that permit the establishment of a twice-daily (bis in diem - b. i. d) dosing regimen and in particular a once daily dosing regimen.

The formulations include those suitable for oral, parenteral (including subcutaneous, intradermal, intramuscular, intravenous and intraaarticular, intranasal, inhalation (including fine particle dusts or mists which may be generated by means of various types of metered dose pressurised aerosols, nebulisers, liquid-based inhalers equipped with appropriate aerolization technologies/apparatus or insufflators), rectal and topical (including dermal, buccal, sublingual and intraocular administration) although the most suitable route may depend upon for example the condition and disorder of the recipient. The formulations may conveniently be presented in unit dosage form and may be prepared by any of the methods well known in the art of pharmacy. All methods include the step of bringing the active ingredients into association with the carrier, which constitutes one or more accessory ingredients/excipients. In general the formulations are prepared by uniformly and intimately bringing into association the active ingredients with liquid carriers or finely divided solid carriers or both and then, if necessary, shaping the product into the desired formulation.

In a preferred embodiment of the invention the pharmaceutical acceptable salt of a syk inhibitor and ciclesonide are provided in form suitable for inhalation. Both active ingredients may be provided in separate dosage forms (free combination) and preferably in a fixed combination.

Formulations for inhalation include powder compositions, which can contain lactose, and spray compositions which may be formulated, for example, as aqueous solutions or suspensions or as aerosols delivered from pressurised packs, with the use of a suitable propellant, e. g. 1,1,1,2-terafluorethane, 1,1,1,2,3,3,3-heptafluoropropane, carbon dioxide or other suitable gas. A class of propellants, which are believed to have minimal ozone-depleting effects in comparison to conventional chlorofluorocarbons comprise hydrofluorocarbons and a number of medidnal aerosol formulations using such propellant systems are disclosed in, for example, EP 0372777, W091/0401 1, W091/11173, W091/11495, W091/14422, W093/11743, and EP-0553298. These applications are all concerned with the preparation of pressurised aerosols for the administration of medicaments and seek to overcome problems associated with the use of this new class of propellants, in particular the problems of stability associated with the pharmaceutical formulations prepared. The applications propose, for example, the addition of one or more of excipients such as polar cosolvents or wetting agents (e.g. alcohols such as ethanol), alkanes, dimethyl ether, surfactants (including fluorinated and non-fluorinated surfactants, carboxylic acids such as oleic acid, polyethoxylates etc.) or bulking agents such as a sugar (see for example W002/30394) and amino acids and vehicles such as cromoglicic acid and/or nedocromil which are contained at concentrations, which are not therapeutically and prophylactically active (see WO00/07567). For suspension aerosols, the active ingredients should be micronised so as to permit inhalation of substantially all of the active ingredients into the lungs upon administration of the aerosol formulation, thus the active ingredients will have a mean particle size of less than 100 microns, desirably less than 20 microns, and preferably in the range 0.7 to 10 microns, for example, 1 to 5 microns.

A suitable formulation for ciclesonide based on hydrofluorocarbon propellants is for example known from U.S. Pat. No. 6,120,752. U.S. Pat. No. 6,120,752 discloses and claims, inter alia, pharmaceutical compositions comprising a therapeutically effective amount of ciclesonide or a related compound and a hydrofluorocarbon propellant, preferably selected from 1,1,1,2-tetrafluoroethane, 1,1,1,2,3,3,3-heptafluoropropane and a mixture thereof, and cosolvent, preferably ethanol, in an amount effective to solubilize ciclesonide and optionally a surfactant.

Canisters generally comprise a container capable of withstanding the vapour pressure of the propellant, such as plastic or plastic-coated glass bottle or a metal can, for example an aluminium can which may optionally be anodised, lacquer-coated and/or plastic-coated, which container is closed with a metering valve. Canisters may be coated with a fluorocarbon polymer as described in WO 96/32150, for example, a co-polymer of polyethersulphone (PES) and polytetrafluoroethylene (PTFE). Another polymer for coating that may be contemplated is FEP (fluorinated ethylene propylene).

The metering valves are designed to deliver a metered amount of the formulation per actuation and incorporate a gasket to prevent leakage of propellant through the valve. The gasket may comprise any suitable elastomeric material such as for example low density polyethylene, chlorobutyl, black and white butadiene-acrylonitrile rubbers, butyl rubber and neoprene. Thermoplastic elastomer valves as described in W092/11190 and valves containing EPDM rubber as described in W095/02650 may be suitable. Suitable valves are commercially available from manufacturers well known in the aerosol industry, for example, from Valois, France (eg. DF10, DF30, DF60), Bespak pic, UK (eg. BK300, BK356, BK357) and 3M-Neotechnic Ltd, UK (eg. Spraymiser). Valve seals, especially the gasket seal and also the seals around the metering chamber, can be manufactured of a material, which is inert to and resists extraction into the contents of the formulation, especially when the contents include ethanol.

Valve materials, especially the material of manufacture of the metering chamber, can be manufactured of a material which is inert to and resists distortion by contents of the formulation, especially when the contents include ethanol. Particularly suitable materials for use in manufacture of the metering chamber include polyesters eg polybutyleneterephthalate (PBT) and acetals, especially PBT. Materials of manufacture of the metering chamber and/or the valve stem may desirably be fluorinated, partially fluorinated or impregnated with fluorine containing substances in order to resist drug deposition.

Valves, which are entirely or substantially composed of metal components (eg Spraymiser, 3M-Neotechnic), are especially preferred for use according to the invention.

Intranasal sprays or nasal drops may be formulated with aqueous or non-aqueous vehicles with or without the addition of agents such as thickening agents, buffer salts or acid or alkali to adjust the pH, isotonicity adjusting agents, preservatives or anti-oxidants. In a preferred embodiment according to the invention the formulation is suitable for topical administration. In a preferred embodiment the formulation according to the invention is a formulation suitable for application to mucosa in the case of treatment of allergic rhinitis. In the case of treatment of allergic conjunctivitis a preferred formulation is a formulation suitable for conjunctival administration (application to the conjunctival sac). The formulations may conveniently be presented in unit dosage form and may be prepared by any of the methods well known in the art of pharmacy. All methods include the step of bringing the active ingredients into association with the carrier, which constitutes one or more accessory ingredients/excipients. In general the formulations are prepared by uniformly and intimately bringing into association the active ingredients with liquid carriers or finely divided solid carriers or both and then, if necessary, shaping the product into the desired formulation.

In a preferred embodiment the present invention relates to an aqueous pharmaceutical composition for the treatment of allergic rhinitis for application to the mucosa, comprising as active ingredients a combination of at least one syk inhibitor and ciclesonide. In particular the aqueous pharmaceutical composition is a sterile aqueous pharmaceutical composition.

The present invention further relates to an aqueous pharmaceutical composition for the treatment of allergic rhinitis for application to the mucosa comprising as active ingredients a combination of at least one syk inhibitor and ciclesonide together with one or more water-insoluble and/or water-low soluble substance and having an osmotic pressure of less than 290 mOsm. Preferably the osmotic pressure is 150 mOsm or lower, more preferably 72 mOsm or lower, more preferably 60 mOsm or lower, more preferably 40 mOsm or lower, more preferably 30 mOsm or lower and still more preferably 20 mOsm (e.g. 10 mOsm or lower).

According to the present invention it is not particularly required to add a substance for controlling osmotic pressure (osmotic pressure-controlling agent) but when it is added any substance can be used. In the present invention, a substance for controlling osmotic pressure (osmotic pressure controlling agent) can be added to control osmotic pressure, specific examples of which include salts such as sodium chloride and water-soluble sugars such as glucose, with glucose being a particularly preferable example.

In a preferred embodiment the pharmaceutical composition is a pharmaceutical composition as described for ciclesonide in U.S. Pat. No. 6,767,901 or WO 01/28563.

Thus in one aspect the present invention relates to an aqueous pharmaceutical composition for the treatment of allergic rhinitis for application to the mucosa, comprising as active ingredients a combination of at least one syk inhibitor and cidesonide together with one or more water-insoluble and/or water-low soluble substance and having an osmotic pressure of less than 290 mOsm.

The water-insoluble or water-low soluble substance may be any substance, and preferred examples include celluloses, more preferably crystalline celluloses and particularly preferred microcrystalline celluloses. According to the present invention, the concentration of water-insoluble and/or water-low soluble substance present in form of solid particles in an aqueous medium is preferably 0.3% w/w and above, and particularly preferably 0.5% w/w to 5% w/w, relative to the total amount of the composition.

In addition, an aqueous polymer substance can also be added in the present pharmaceutical composition. Specific examples of such include propylene glycol alginate, pectin, low methoxyl pectin, gua gum, gum Arabic, carrageenan, methyl cellulose, carboxymethyl cellulose sodium, xanthan gum hydroxypropylmethyl cellulose and hydroxypropyl cellulose, while particularly preferable examples include carboxymethyl cellulose sodium, polyethylene glycol and hydroxypropyl cellulose. Carboxymethyl cellulose sodium blended with microcrystalline cellulose, is an example of a combination of these water-soluble substance and water-insoluble substance that can be used in the present invention. Furthermore, in the case of adding these water-soluble polymer substances, the concentration of said substance is preferably 1% w/w to 30% w/w relative to the water-insoluble substance and/or water-low soluble substance.

In a preferred embodiment of the invention hydroxypropylmethyl cellulose is contained in the pharmaceutical compositions according to the invention. The hydroxypropylmethyl cellulose may be any grade, a specific example is hydroxypropylmethyl cellulose 2910. Although said hydroxypropylmethyl cellulose may be present at any concentration, its concentration is preferably from 0.001% w/w to 30 % w/w, particularly preferably form 0.01% w/w to 5% w/w, more particularly preferably from 0.01% w/w to 1% w/w, and most preferably from 0.01% w/w to 0.5% w/w, relative to the total amount of composition.

A surfactant and/or wetting agent, although not essential in the present invention, can be added, specific examples of which include Polysorbate 80, glycerin monosterarate, polyoxyl stearate, lauromacrogol, sorbitan oleate and sucrose fatty acid esters.

In another embodiment of the invention the pharmaceutical formulation comprising the syk inhibitor in combination with ciclesonide is a dry powder, i.e. ciclesonide and the syk inhibitor are present in a dry powder comprising finely divided pharmaceutical acceptable salt of the syk inhibitor and ciclesonide optionally together with a finely divided pharmaceutically acceptable carrier, which is preferably present and may be one or more materials known as carriers in dry powder inhalation compositions, for example saccharides, including monosaccharides, disaccharides, polysaccharides and sugar alcohols such as arabinose, glucose, fructose, ribose, mannose, sucrose, trehalose, lactose, maltose, starches, dextran or mannitol. An especially preferred carrier is lactose, particularly in the form of the monohydrate. The dry powder may be in capsules of gelatine or plastic, or in blisters, for use in a dry powder inhalation device, preferably in dosage units of the mixture of a syk inhibitor and ciclesonide together with the carrier in amounts to bring the total weight of powder in each capsule to from 5mg to 50mg. Alternatively the dry powder may be contained in a reservoir of a multi-dose dry powder inhalation device. Capsules and cartridges of for example gelatin, or blisters of for example laminated aluminium foil, for use in an inhaler or insulator may be formulated containing a powder mix of the active ingredients and a suitable powder base such as lactose or starch, preferably lactose. In this aspect, the active ingredients are suitably micronised so as to permit inhalation of substantially all of the active ingredients into the lungs upon administration of the dry powder formulation, thus the active ingredients will have a particle size of less than 100 μpm, desirably less than 20 μm, and preferably in the range 1 to 10 μm. The solid carrier, where present, generally has a maximum particle diameter of 300 μm, preferably 200 μm, and conveniently has a mean particle diameter of 40 to 100 μm, preferably 50 to 75 μm. The particle size of the active ingredients and that of a solid carrier where present in dry powder compositions, can be reduced to the desired level by conventional methods, for example by grinding in an air-jet mill, ball mill or vibrator mill, microprecipitation, spray drying, lyophilisation or recrystallisation from supercritcal media.

Where the inhalable form of the composition of the invention is the finely divided particulate form, the inhalation device may be, for example a dry powder inhalation device adapted to deliver dry powder from a capsule or blister containing a dosage unit of the dry powder or a multi-dose dry powder inhalation device. Such dry powder inhalation devices are known in the art. Examples which may be mentioned are Cyclohaler®, Diskhaler® Rotadisk®, Turbohaler®, Novolizer® or the dry powder inhalation devices disclosed EP 0 505 321, EP 407028, EP 650410, EP 691865 or EP 725725 (Ultrahaler®) .

Formulations for inhalation by nebulization may be formulated with an aqueous vehicle with the addition of agents such as alcohols, acid or alkali, buffer salts, isotonicity adjusting agents or antimicrobials. They may be sterilised by filtration or heating in an autoclave. Suitable technologies for this type of administration are known in the art. As an example the Mystic® technology is to be mentioned (see for example U.S. Pat. Nos. 6,397,838; 6,454,193; and 6,302,331) as well as Respimat® and the e-flow (Pari).

Preferred unit dosage formulations are those containing a pharmaceutical effective dose, as hereinbefore recited, or an appropriate fraction thereof, of the active ingredient. Thus, in the case of formulations designed for delivery by metered dose pressurised aerosols, one actuation of the aerosol may deliver half of the therapeutical effective amount such that two actuations are necessary to deliver the therapeutically effective dose.

It should be understood that in addition to the ingredients particularly mentioned above, the formulations of this invention may include other agents conventional in the art having regard to the type of formulation in question. Furthermore, the claimed formulations include bioequivalents as defined by the US Food and Drug Administration.

All patents, patent applications, and publications cited herein are hereby incorporated by reference in their entirety.

The invention will now be illustrated by the following examples without restricting it.

A. Fixed Combinations

Ciclesonide and syk inhibitor aqueous pharmaceutical compositons containing the components indicated below can be prepared by processing with a homomixer. Homomixer processing is performed, e.g., at 6000 rpm for 30 minutes.

### EXAMPLE 1

### Intranasal Formulation Combination of Ciclesonide and Syk Inhibitor

| | |
|---|---|
| Ciclesonide: | 0.05% |
| Syk Inhibitor | appropriate amount |
| Microcrystalline cellulose and | 1.7% |
| carboxymethyl cellulose sodium | |
| Hydroxypropylmethyl cellulose 2910 | 0.1% |

B. Free Combinations

### EXAMPLE 1

### Ciclesonide is Provided as Pharmaceutical Product Comprising an Aerosol Vial Equipped with a Dispensing Valve and Containing the Following Formulation

| | | |
|---|---|---|
| Ciclesonide | 1.000 | mg/ml |
| Ethanol | 94.800 | mg/ml |
| P134a | 1090.200 | mg/ml |

### EXAMPLE 2

### Syk Inhibitor Suspension Formulation Suitable for Nasal Administration

0.5-20 mg/ml syk inhibitor
0.1-0.2 mg/ml benzalkonium chloride
0.5-5 mg/ml polysorbate 80
1-15 mg/ml microcrystalline cellulose or carboxymethylcellulose sodium
1-4 mg/ml phenylethanol
20-50 mg/ml dextrose
pH adjusted to pH 5.5

### EXAMPLE 3

### Syk Inhibitor Suspension Formulation Suitable for Inhalation Administration

1-20 mg/ml syk inhibitor
0.1-1% polysorbate 80
50 mm citrate and/or 0.9% sodium chloride

Although the invention has been described in terms of specific formulations and ingredients, it will be understood that these are not intended to be limiting. To the contrary, those skilled in the art will understand that various optional ingredients may be included, such as flavouring agents, preservatives, additional active ingredients, and the like, while still embodying the present invention.

1. A pharmaceutical formulation comprising a syk inhibitor in combination with ciclesonide, or a pharmaceutically acceptable salt, solvate or physiologically functional derivative thereof and a pharmaceutically acceptable carrier and/or one or more excipients, and optionally one or more other therapeutic ingredients.

2. The formulation according to claim 1, wherein the syk inhibitor and ciclesonide are contained in the same pharmaceutical formulation (fixed combination).

3. The formulation according to claim 1, wherein the syk inhibitor and ciclesonide are contained in different pharmaceutical formulations (free combination).

4. The formulation according to claim 1, comprising a compound selected from the group consisting of [11β,16α(R)]-16,17-[(Cyclohexylmethylen)bis(oxy)]-11-hydroxy-21-(2-methyl-1-oxopropoxy)pregna-1,4-dien-3,20-dion, [11β,16α(S)]-16,17-[(Cyclohexylmethylen)bis(oxy)]-11-hydroxy-21-(2-methyl-1-oxoprop-oxy)pregna-1,4-dien3,20-dion, [11β,16α-(R,S)]-16,17-[(Cyclohexyl-methylen)bis(oxy)]-11-hydroxy-21-(2-methyl-1-oxoprop-oxy)pregna-1,4-dien3,20-dion, 16α,17-(22R)-Cyclohexylmethylendioxy-11β,21-dihydroxy-pregna-1,4-dien-3,20-dion, 16α,17-(22S)-Cyclohexylmethylendioxy-11β,21-dihydroxypregna-1,4-dien-3,20-dion and 16α,17-(22R,S)-Cyclohexylmethylendioxy-11β,21-dihydroxypregna-1,4-dien3,20-dion.

5. The formulation according to claim 1, comprising a syk inhibitor and ciclesonide in an amount and ratio to be effective for a twice or once daily treatment of a clinical condition in a mammal for which a corticosteroid and/or a syk inhibitor is indicated.

6. The formulation according to claim 1, which is suitable for administration by inhalation.

7. The formulation according to claim 1, which is suitable for nasal administration.

8. The pharmaceutical formulation according to claim 1, which is a dry powder and the carrier is a saccharide.

9. The pharmaceutical formulation according to claim 8, wherein the carrier is lactose monohydrate.

10. A method for the prophylaxis or treatment of a clinical condition in a mammal for which a corticosteroid and/or a syk inhibitor is indicated, which comprises simultaneous or sequential administration of a therapeutically effective amount of ciclesonide or a pharmaceutical acceptable salt, solvate, or physiologically functional derivative thereof in combination with a syk inhibitor.

11. The method according to claim 10, wherein the clinical condition is selected from the group consisting of asthma, nocturnal asthma, exercise-induced asthma, chronic obstructive pulmonary diseases (COPD), chronic bronchitis, wheezy bronchitis, emphysema, shortness of breath, respiratory tract infection, upper respiratory tract disease, rhinitis, allergic rhinitis and seasonal rhinitis.

12. The method according to claim 11, which comprises a twice-daily dosage regimen.

13. The method according to claim 11, which comprises a once daily dosage regimen.

14. The method according to claim 11, which comprises administration of a combination of a syk inhibitor and ciclesonide in the same administration form by inhalation from an inhaler and wherein each actuation provides a dose therapeutically effective for a twice daily dosing regiment or for a once daily dosing regimen.

15. The method according to claim 11, which comprises administration of a combination of a syk inhibitor and ciclesonide in the same administration form by nasal administration.

16. The formulation according to claim 1, wherein the syk inhibitor is selected from the group consisting of 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide, 2-(cis-2-aminocyclohexylamino)-4-(3,4,5-trimeth-oxyanilino)pyrimidine-5-carboxamide and salts thereof.

17. The formulation according to claim 1, wherein the syk inhibitor is selected from the group consisting of N2,N4-[(2,2-Dimethyl-4H-benzo[1,4]oxazin-3-one)-6-yl]-5-fluoro-2,4-pyrimidinediamine,
N4-(3,4-Dichlorophenyl)-5-fluoro-N2-(indazoline-6-yl)-2,4-pyrimidinediamine,
N4-(3,4-Ethylenedioxyphenyl)-5-fluoro-N2-(1-methyl-indazoline-5-yl)-2,4-pyrimidinediamine,
N2,N4-Bis(3-hydroxyphenyl)-5-fluoro-2,4-pyrimidinediamine,
N2,N4-Bis(3,4-ethylenedioxyphenyl)-5-fluoro-2,4-pyrimidinediamine,
N4-(1,4-Benzoxazin-6-yl)-5-fluoro-N2-[3-(N-methylamino)carbonylmethyleneoxyphenyl ]-2,4-pyrimidinediamine,
N2,N4-Bis(3-aminophenyl)-5-fluoro-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N4-(3-Hydroxyphenyl)-5-trifluoromethyl-N2-[3-(N-methylamino)carbonylmethyleneoxyphenyl]-2,4-pyrimidinediamine,
5-Fluoro-N4-[(1H)-indol-6-yl]-N2-[3-(N-methylamino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
5-Fluoro-N2-(3-methylaminocarbonylmethyleneoxyphenyl)-N4-[2-H-pyrido[3,2-b]-1,4-oxazin-3(4H)-one-6-yl]-2,4-pyrimidinediamine,
N4-(3,4-Ethylenedioxyphenyl)-5-fluoro-N2-[3-(2-hydroxyethyl-amino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N2,N4-Bis(indol-6-yl)-5-fluoro-2,4-pyrimidinediamine,
5-Fluoro-N2-[2-(2-hydroxy-1,1-dimethylethylamino)carbonylbenzofuran-5-yl]-N4-(3-hydroxyphenyl)-2,4-pyrimidinediamine,
N2-[3-(N2,3-Dihydroxypropylamino)carbonylmethyleneoxyphenyl]-N4-(3,4-ethylenedioxyphenyl)-5-fluoro-2,4-pyrimidinediamine,
N2-(3,5-Dimethoxyphenyl)-N4-(3,4-ethylenedioxyphenyl)-5-fluoro-2,4-pyrimidinediamine,
N4-(3,4-Ethylenedioxyphenyl)-5-fluoro-N2-[3-(1,3-oxazol-5-yl)phenyl]-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N2-(3-hydroxyphenyl)-N4-[4-(3-phenyl-1,2-4-oxadiazol-5-yl)methyleneoxyphenyl]-2,4-pyrimidinediamine,
N4-(3,4-Ethylenedioxyphenyl)-5-fluoro-N2-(indazolin-6-yl)-2,4-pyrimidinediamine,
5-Fluoro-N4-(3-hydroxyphenyl)-N2-(indazolin-6-yl)-2,4-pyrimidinediamine,
N4-(3,4-Ethylenedioxyphenyl)-5-fluoro-N2-(1-methyl-indazoline-5-yl)-2,4-pyrimidinediamine,
5-Fluoro-N4-(3-hydroxyphenyl)-N2-(1-methy-indazoline-5-yl)-2,4-pyrimidinediamine,
N4-(3,4-Ethylenedioxyphenyl)-5-fluoro-N2-[4-(3-phenyl-1,2,4-oxadiazol-5-yl)methyleneoxyphenyl]-2,4-pyrimidinediamine,
N4-(3,5-Dimethyl-4-hydroxyphenyl)-5-fluoro-N2-[3-[2-(N-morpholino)ethyleneoxy]phenyl]-2,4-pyrimidinediamine,
N4-(3,5-Dimethyl-4-hydroxyphenyl)-5-fluoro-N2-[3-[2-(N-morpholino)ethyloxy]phenyl]-2,4-pyrimidinediamine,
N4-(3-Chloro-4-hydroxy-5-methylphenyl)-5-fluoro-N2-[3-[2-(N-morpholino)ethyloxy]phenyl]-2,4-pyrimidinediamine,
N2-(3-tert-Butylcarbonylaminophenyl)-N4-(3-hydroxyphenyl)-5-fluoro-2,4-pyrimidinediamine,
N4-(3-tert-Butylphenyl)-N2-[3-(N-methylamino)carbonylmethyleneoxyphenyl]-5-fluoro-2,4-pyrimidinediamine,
N4-(3-tert-Butylphenyl)-N2-[3-(N2,3-dihydroxypropylamino)carbonylmethyleneoxyphenyl]-5-fluoro-2,4-pyrimidinediamine,
N2-[3-(N2,3-Dihydroxypropylamino)-carbonylmethyleneoxyphenyl]-5-fluoro-N4-(3-isopropylphenyl)-2,4-pyrimidinediamine,
N4-[4-(Cyanomethyleneoxy)phenyl]-5-fluoro-N2-(3-hydroxyphenyl)-2,4-pyrimidinediamine,
N4-(3,5-Dimethyl-4-hydroxyphenyl)-5-fluoro-N2-[3-(N-piperazino)carbonylmethyleneoxyphenyl]-2,4-pyrimidinediamine,
N4-(3,5-Dimethyl-4-hydroxyphenyl)-5-fluoro-N2-[3-[2-(N-piperazino)ethoxy]phenyl]-2,⁴-pyrimidinediamine bis hydrogenchloride salt,
N4-(3,4-Ethylenedioxyphenyl)-5-fluoro-N2-[4-(2-hydroxyethyloxy)phenyl]-2,4-pyrimidinediamine,
N4-(1,4-Benzoxazine-3-on-6-yl)-5-fluoro-N2-(3-hydroxyphenyl)-2,4-pyrimidinediamine,
(+/−)-5-Fluoro-N2-[(N-methylacetamido-2)-3-phenoxy]-N4-(2-methyl-1,4-benzoxazin-6-yl)-2,4-pyrimidinediamine,
N2-(1,4-Benzoxazin-3-on-6-yl)-5-fluoro-N4-(3-hydroxyphenyl)-2,4-pyrimidinediamine,
N4-(3-Chloro-4-trifluoromethoxyphenyl)-5-fluoro-N2-[3-(N-methylamino)carbonylmethyleneoxyphenyl]-2,4-pyrimidinediamine,
5-Fluoro-N4-(3-hydroxy-4-methylphenyl)-N2-[3-[(N-methylamino)carbonylmethyleneoxy]phenyl]-2,4-pyrimidinediamine,
5-Fluoro-N4-(3-hydroxyphenyl)-N2-[4-methyl-3-[(N-methylamino)carbonylmethyleneoxy]phenyl]-2,4-pyrimidinediamine,
5-Fluoro-N4-(3-hydroxy-4-methoxyphenyl)-N2-[3-(N-methylamino)carbonylmethyleneoxyphenyl]-2,4-pyrimidinediamine,
N4-(3-Chloro-4-methylphenyl)-5-fluoro-N2-[3-(N-methylamlno)-carbonylmethyleneoxyphenyl]-2,4-pyrimidinediamine,
N4-(3-Chloro-4-methoxyphenyl)-5-fluoro-N2-[3-[(N-methylamino)carbonylmethyleneoxy]phenyl]-2,4-pyrimidinediamine,
5-Fluoro-N4-1(1H)-indol-5-yl]-N2-[3-[(N-methylamino)carbonylmethyleneoxy]phenyl]-2,4-pyrimidinediamine,
5-Fluoro-N4-(3-hydroxyphenyl)-N2-[1-(methoxycarbonyl)methyl-indazoline-5-yl]-2,4-pyrimidinediamine,
5-Fluoro-N4-(3-hydroxyphenyl)-N2-[1-(3-hydroxypropyl)indazoline-6-yl]-2,4-pyrimidinediamine,
N4-(3,4-Ethylenedioxyphenyl)-5-fluoro-N2-[1-(3-hydroxypropyl)indazoline-5-yl]-2,4-pyrimidinediamine,
5-Fluoro-N4-(3-hydroxyphenyl)-N2-[1-(3-hydroxypropyl)indazoline-5-yl]-2,4-pyrimidinediamine,
5-Fluoro-N2-[1-(3-hydroxypropyl)indazoline-5-yl]-N4-(4-isopropoxyphenyl)-2,4-pyrimidinediamine,
N4-(3,4-Ethylenedioxyphenyl)-5-fluoro-N2-[1-[2(N-methylaminocarbonyl)ethyl]-indazoline-5-yl]-2,4-pyrimidinediamine,
5-Fluoro-N4-(4-isopropoxyphenyl)-N2-[1-[2(N-methylaminocarbonyl)ethyl]-indazoline-5-yl]-2,4-pyrimidinediamine,
N4-[(2,2-dimethyl-4H-benzo[1,4]oxazin-3-one)-6-yl]-5-fluoro-N2-[3-(methylaminocarbonylmethyleneoxy)phenyl]-2,4-pyrimidinediamine,
N4-[(2,2-Dimethyl-4H-benzo[1,4]oxazin-3-one)-6-yl]-5-fluoro-N2-(1-methylindazolin-5-yl)-2,4-pyrimidinediamine,
N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine,
N4-1(2,2-Dimethyl-4H-5-pyridol-1,4]oxazin-3-one)-6-yl]-5-fluoro-N2-[3-(methylaminocarbonylmethyleneoxy)phenyl]-2,4-pyrimidinediamine,
5-Fluoro-N2-(3-methylaminocarbonylmethyleneoxyphenyl)-N4-[2H-pyrido[3,2-b]-1,4-oxazin-3(4H)-one-6-yl]-2,4-pyrimidinediamine,
N4-(4-Amino-3,4-dihydro-2H-1-benzopyran-6-yl)-5-fluoro-N2-[3-(N-methylamino)carbonylmethylene oxyphenyl]-2,4-pyrimidinediamine,
N4-(3-Methylcarbonyloximephenyl)-5-fluoro-N2-[3-(N-methylamino)carbonylmethyleneoxyphenyl]-2,4-pyrimidinediamine and salts thereof.

18. The method according to claim 10, wherein the syk inhibitor is selected from the group consisting of 2-(2-aminoethylamino)-4-(3-methylanilino)pyrimidine-5-carboxamide, 2-(2-aminoethylamino)-4-(3-trifluoromethylanilino)pyrimidine-5-carboxamide, 2-(4-aminobutylamino)-4-(3-trifluoromethylanilino)pyrimidine-5-carboxamide, 2-(2-aminoethylamino)-4-(3-bromoanilino)pyrimidine-5-carboxamide, 2-(2-aminoethylamino)-4-(3-nitroanilino)pyrimidine-5-carboxamide, 2-(2-aminoethylamino)-4-(3,5-dimethylanilino)pyrimidine-5-carboxamide, 2-(2-aminoethylamino)-4-(2-naphthylamino)pyrimidine-5-carboxamide, 2-(cis-2-aminocyclohexylamino)-4-(3-methylanilino)pyrimidine-5-carboxamide, 2-(cis-2-aminocyclohexylamino)-4-(3-bromoanilino)pyrimidine-5-carboxamide, 2-(cis-2-aminocyclohexylamino)-4-(3,5-dichloroanilino)pyrimidine-5-carboxamide, 2-(cis-2-aminocyclohexylamino)-4-(3,4,5-trimethoxyanilino)pyrimidine-5-carboxamide and salts thereof.

19. The method according to claim 10, wherein the syk inhibitor is selected from the group consisting of
N2,N4-[(2,2-Dimethyl-4H-benzo[1,4]oxazin-3-one)-6-yl]-5-fluoro-2,4-pyrimidinediamine,
N4-(3,4-Dichlorophenyl)-5-fluoro-N2-(indazoline-6-yl)-2,4-pyrimidinediamine,
N4-(3,4-Ethylenedioxyphenyl)-5-fluoro-N2-(1-methyl-indazoline-5-yl)-2,4-pyrimidinediamine,
N2,N4-Bis(3-hydroxyphenyl)-5-fluoro-2,4-pyrimidinediamine,
N2,N4-Bis(3,4-ethylenedioxyphenyl)-5-flubro-2,4-pyrimidinediamine,
N4-(1,4-Benzoxazin-6-yl)-5-fluoro-N2-[3-(N-methylamino)carbonylmethyleneoxyphenyl]-2,4-pyrimidinediamine,
N2,N4-Bis(3-aminophenyl)-5-fluoro-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N4-(3-Hydroxyphenyl)-5-trifluoromethyl-N2-[3-(N-methylamino)carbonylmethyleneoxyphenyl]-2,4-pyrimidinediamine,
5-Fluoro-N4-[(1H)-indol-6-yl]-N2-[3-(N-methylamino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
5-Fluoro-N2-(3-methylaminocarbonylmethyleneoxyphenyl)-N4-[2-H-pyrido[3,2-b]-1,4-oxazin-3(4H)-one-6-yl]-2,4-pyrimidinediamine,
N4-(3,4-Ethylenedioxyphenyl)-5-fluoro-N2-[3-(2-hydroxyethyl-amino)carbonylmethyleneoxyphenyl]-2,4-pyrimidinediamine,
5-Fluoro-N4-(3-hydroxyphenyl)-N2-[3-(N-methylamino)carbonylmethyleneoxyphenyl]-2,4-pyrimidinediamine,
N2,N4-Bis(indol-6-yl)-5-fluoro-2,4-pyrimidinediamine,
5-Fluoro-N2-[2-(2-hydroxy-1,1-dimethylethylamino)carbonylbenzofuran-5-yl]-N4-(3-hydroxyphenyl)-2,4-pyrimidinediamine,
N2-[3-(N2,3-Dihydroxypropylamino)carbonylmethyleneoxyphenyl]-N4-(3,4-ethylenedioxyphenyl)-5-fluoro-2,4-pyrimidinediamine,
N2-(3,5-Dimethoxyphenyl)-N4-(3,4-ethylenedioxyphenyl)-5-fluoro-2,4-pyrimidinediamine,
N4-(3,4-Ethylenedioxyphenyl)-5-fluoro-N2-[3-(1,3-oxazol-5-yl)phenyl]-2,4-pyrimidinediamine,
N4-(3,4-Ethylenedioxyphenyl)-5-fluoro-N2-[3-(N-methylamino)-carbonylmethyleneoxyphenyl]-2,4-pyrimidinediamine,
5-Fluoro-N2-(3-hydroxyphenyl)-N4-[4-(3-phenyl-1,2-4-oxadiazol-5-yl)methyleneoxyphenyl]-2,4-pyrimidinediamine,
N4-(3,4-Ethylenedioxyphenyl)-5-fluoro-N2-(indazolin-6-yl)-2,4-pyrimidinediamine,
5-Fluoro-N4-(3-hydroxyphenyl)-N2-(indazolin-6-yl)-2,4-pyrimidinediamine,
N4-(3,4-Ethylenedioxyphenyl)-5-fluoro-N2-(1-methyl-indazoline-5-yl)-2,4-pyrimidinediamine,
5-Fluoro-N4-(3-hydroxyphenyl)-N2-(l-methyl-indazoline-5-yl)-2,4-pyrimidinediamine,
N4-(3,4-Ethylenedioxyphenyl)-5-fluoro-N2-[4-(3-phenyl-1,2,4-oxadiazol-5-yl)methyleneoxyphenyl]-2,4-pyrimidinediamine,
N4-(3,5-Dimethyl-4-hydroxyphenyl)-5-fluoro-N2-[3-[2-(N-morpholino)ethyleneoxy]phenyl]-2,4-pyrimidinediamine,
N4-(3,5-Dimethyl-4-hydroxyphenyl)-5-fluoro-N2-[3-[2-(N-morpholino)ethyloxy]phenyl]-2,4-pyrimidinediamine,
N4-(3-Chloro-4-hydroxy-5-methylphenyl)-5-fluoro-N2-[3-[2-(N-morpholino)ethyloxy]phenyl]-2,4-pyrimidinediamine,
N2-(3-tert-Butylcarbonylaminophenyl)-N4-(3-hydroxyphenyl)-5-fluoro-2,4-pyrimidinediamine,
N4-(3-tert-Butylphenyl)-N2-[3-(N-methylamino)carbonylmethyleneoxyphenyl]-5-fluoro-2,4-pyrimidinediamine,
N4-(3-tert-Butylphenyl)-N2-[3-(N2,3-dihydroxypropylamino)carbonylmethyleneoxyphenyl]-5-fluoro-2,4-pyrimidinediamine,
N2-[3-(N2,3-Dihydroxypropylamino)carbonylmethyleneoxyphenyl]-5-fluoro-N4-(3-isopropylphenyl)-2,4-pyrimidinediamine,
N4-[4-(Cyanomethyleneoxy)phenyl]-5-fluoro-N2-(3-hydroxyphenyl)-2,4-pyrimidinediamine,
N4-(3,5-Dimethyl-4-hydroxyphenyl)-5-fluoro-N2-[3-(N-piperazino)carbonylmethyleneoxyphenyl]-2,4-pyrimidinediamine,
N4-(3,5-Dimethyl-4-hydroxyphenyl)-5-fluoro-N2-[3-[2-(N-piperazino)ethoxy]phenyl]-2,4-pyrimidinediamine bis hydrogenchloride salt,
N4-(3,4-Ethylenedioxyphenyl)-5-fluoro-N2-[4-(2-hydroxyethyloxy)phenyl]-2,4-pyrimidinediamine,
N4-(1,4-Benzoxazine-3-on-6-yl)-5-fluoro-N2-(3-hydroxyphenyl)-2,4-pyrimidinediamine,
(+/−)-5-Fluoro-N2-[(N-methylacetamido-2)-3-phenoxy]-N4-(2-methyl-1,4-benzoxazin-6-yl)-2,4-pyrimidinediamine,
N2-(1,4-Benzoxazin-3-on-6-yl)-5-fluoro-N4-(3-hydroxyphenyl)-2,4-pyrimidinediamine,
N4-(3-Chloro-4-trifluoromethoxyphenyl)-5-fluoro-N2-[3-(N-methylamino)carbonylmethyleneoxyphenyl]-2,4-pyrimidinediamine,
5-Fluoro-N4-(3-hydroxy-4-methylphenyl)-N2-[3-[(N-methylamino)carbonylmethyleneoxy]phenyl]-2,4-pyrimidinediamine,
5-Fluoro-N4-(3-hydroxyphenyl)-N2-[4-methyl-3-[(N-methylamino)carbonylmethyleneoxy]phenyl]-2,4-pyrimidinediamine,
5-Fluoro-N4-(3-hydroxy-4-methoxyphenyl)-N2-[3-(N-methylamino)carbonylmethyleneoxyphenyl]-2,4-pyrimidinediamine,
N4-(3-Chloro-4-methylphenyl)-5-fluoro-N2-[3-(N-methylamino)-carbonylmethyleneoxyphenyl]-2,4-pyrimidinediamine,
N4-(3-Chloro-4-methoxyphenyl)-5-fluoro-N2-[3-[(N-methylamino)carbonylmethyleneoxy]phenyl]-2,4-pyrimidinediamine,
5-Fluoro-N4-1(1H)-indol-5-yl]-N2-[3-[(N-methylamino)carbonylmethyleneoxy]phenyl]-2,4-pyrimidinediamine,
5-Fluoro-N4-(3-hydroxyphenyl)-N2-[1-(methoxycarbonyl)methyl-indazoline-5-yl]-2,4-pyrimidinediamine,
5-Fluoro-N4-(,3-hydroxyphenyl)-N2-[1-(3-hydroxypropyl)indazoline-6-yl]-2,4-pyrimidinediamine,
N4-(3,4-Ethylenedioxyphenyl)-5-fluoro-N2-[1-(3-hydroxypropyl)indazoline-5-yl]-2,4-pyrimidinediamine,
5-Fluoro-N4-(3-hydroxyphenyl)-N2-[1-(3-hydroxypropyl)indazoline-5-yl]-2,4-pyrimidinediamine,
5-Fluoro-N2-[1-(3-hydroxypropyl)indazoline-5-yl]-N4-(4-isopropoxyphenyl)-2,4-pyrimidinediamine,
N4-(3,4-Ethylenedioxyphenyl)-5-fluoro-N2-[1-[2(N-methylaminocarbonyl)ethyl]-indazoline-5-yl]-2,4-pyrimidinediamine,
5-Fluoro-N4-(4-isopropoxyphenyl)-N2-[1-[2(N-methylaminocarbonyl)ethyl]-indazoline-5-yl]-2,4-pyrimidinediamine,
N4-((2,2-dimethyl-4H-benzo[1,4]oxazin-3-one)-6-yl]-5-fluoro-N2-[3-(methylaminocarbonylmethyleneoxy)phenyl]-2,4-pyrimidinediamine,
N4-[(2,2-Dimethyl-4H-benzo[1,4]oxazin-3-one)-6-yl]-5-fluoro-N2-(1-methylindazolin-5-yl)-2,4-pyrimidinediamine,
N4-[(2,2-Difluoro-4H-benzo[1,4]oxazin-3-one)-6-yl]-5-fluoro-N2-[3-(methylaminocarbonylmethyleneoxy)phenyl]-2,4-pyrimidinediamine,
N4-1(2,2-Dimethyl-4H-5-pyridol-1,4]oxazin-3-one)-6-yl]-5-fluoro-N2-[3-(methylaminocarbonylmethyleneoxy)phenyl]-2,4-pyrimidinediamine,
5-Fluoro-N2-(3-methylaminocarbonylmethyleneoxyphenyl)-N4-[2H-pyrido[3,2-b]-1,4-oxazin-3(4H)-one-6-yl]-2,4-pyrimidinediamine,
N4-(4-Amino-3,4-dihydro-2H-1-benzopyran-6-yl)-5-fluoro-N2-[3-(N-methylamino)carbonylmethyleneoxyphenyl]-2,4-pyrimidinediamine,
N4-(3-Chloro-4-hydroxy-5-methylphenyl)-5-fluoro-N2-[3-[2-(N-piperazino)ethoxy]phenyl]-2,4-pyrimidinediamine,
N4-(3-Methylcarbonyloximephenyl)-5-fluoro-N2-[3-(N-methylamino)carbonylmethyleneoxyphenyl]-2,4-pyrimidinediamine and salts thereof.
